(19) United States
(12) Patent Application Publication
(10) Pub. No.: US 2009/0076268 A1
(43) Pub. Date: Mar. 19, 2009

(54) Facile assembly of fused benzofuro-heterocycles

(76) Inventors: Anne E. Fitzgerald, San Diego, CA (US); Jing Liu, San Diego, CA (US); Neelakandha S. Mani, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/283,604

(22) Filed: Sep. 12, 2008

Related U.S. Application Data
(60) Provisional application No. 60/972,357, filed on Sep. 14, 2007.

(51) Int. Cl. C07D 491/048; C07D 307/91

(52) U.S. Cl. 544/250; 549/460; 549/461; 546/89

(57) ABSTRACT

This invention concerns the synthesis of polycyclic structural components of pharmacological compounds, including the synthesis of fused benzofuro-heterocycles, through selective palladium-catalyzed cross-coupling and intramolecular cyclization.

This application claims the benefit of U.S. provisional patent application Ser. No. 60/972,357, filed on Sep. 14, 2007, which is incorporated herein by reference in its entirety.

### FIELD OF THE INVENTION

This invention concerns the synthesis of polycyclic structural components of pharmacological compounds, including the synthesis of fused benzofuro-heterocycles, through halogen-selective Suzuki cross-coupling and intramolecular cyclization reactions.

### BACKGROUND OF THE INVENTION

Fused benzofuro-heterocycles are common structural motifs in biologically important natural products and drug candidates.

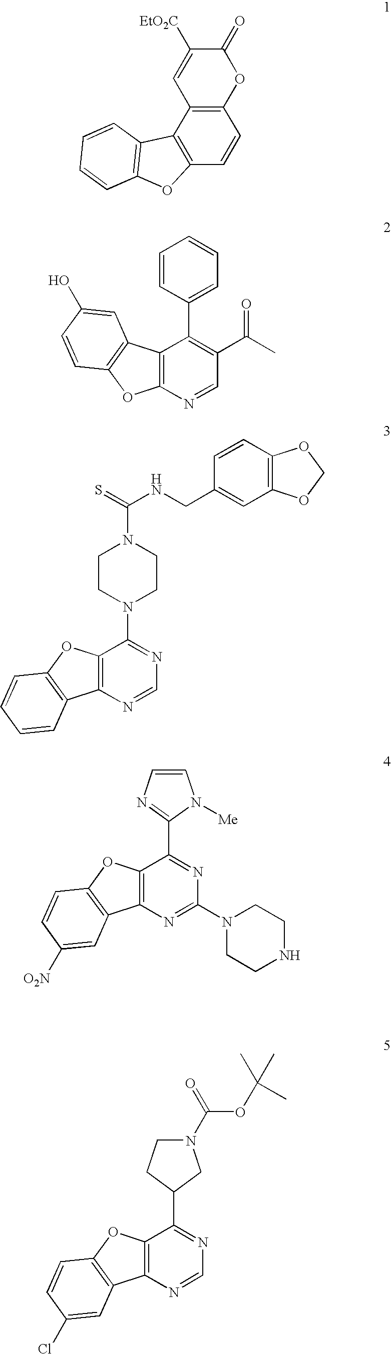

For example, benzofurocoumarins such as 1 were found to inhibit the growth of human cancer cell lines.¹ Elbfluorene (2) and its derivatives are interesting leads as cyclin-dependent kinase (CDK) inhibitors.² Benzofuropyrimidine 3 (MP-470, SuperGen, Inc., Dublin, Calif.) is a novel multitarget tyrosine kinase inhibitor currently in Phase I clinical trials,³ while compound 4 and its analogs exhibit potent blood sugar-lowering activity without inducing low blood sugar or increasing blood lactic acid.⁴ Benzofuropyrimidine is also the key structural core of a group of histamine H₄ receptor modulators such as 5.⁵

In the literature, benzofuro-heterocycles 6 are sometimes prepared by the intramolecular cyclization of 2′-halobiophenyl-2-ol (7) as depicted in Scheme S.

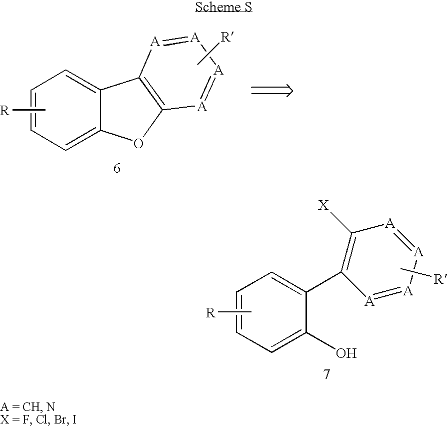

For example, benzofuropyridines have been prepared from the intramolecular cyclization of the corresponding phenols, which are synthesized via base-catalyzed rearrangement of N-hydroxypyridinium salts.⁶ Li and coworkers reported the preparation of all four benzofuropyridine regioisomers using four different routes, two of which involve tandem Stille coupling/intramolecular cyclization.⁷ Very recently, the synthesis of ladder-type heteroacenes containing dibenzofuran moieties via sequential Suzuki coupling and O-arylation was reported.⁸ Also, there are a few isolated cases of synthesis of dibenzofurans⁹ and benzofuropyrazines¹⁰ from the corresponding biaryl phenols. These literature examples have several limitations. Many of them are lengthy multi-step syntheses, and the intramolecular cyclization reactions usually require harsh conditions such as strong base and high temperature. In addition, these methods have limited substrate scope and cannot be considered a general approach for the preparation of fused benzofuro-heterocylic compounds.

In contrast to the literature metholodogy, the present invention provides a general route to a wide variety of benzofuroheterocycles. The present invention provides novel methodologies for the preparation of fused benzofuro-heterocycles, such as dibenzofurans, benzofuropyridines, benzofuropyrimidines, and benzofuropyrazines, from halogen-selective Suzuki coupling of aryl boronic acid derivatives with haloarenes, an optional deprotection step, and subsequent intramolecular cyclization. These reactions present useful methods for the synthesis of these complex heterocyclic systems.

### SUMMARY OF THE INVENTION

In one aspect the invention relates to a process for the preparation of a compound of Formula (I) or salts thereof:

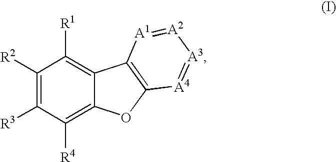

wherein
R¹, R², R³, and R⁴ are each independently H, fluoro, chloro, bromo, C₁₋₄alkyl, —OC₁₋₄alkyl, —CF₃, —OCF₃, —CN, —NO₂, —S(O)C₁₋₄alkyl, —SO₂C₁₋₄alkyl, —CHO, —C(O)C₁₋₄alkyl, —CO₂C₁₋₄alkyl, —CO₂H, —C(O)NR^{a}R^{b}, or —NR^{a}R^{b};
   where R^{a} and R^{b} are each independently C₁₋₄alkyl;
A¹, A², A³, and A⁴ are each independently CR^{c} or N;
   where at least two of A¹⁻⁴ are CR^{c}; and
   each R^{c} is independently H, fluoro, chloro, bromo, C₁₋₄alkyl, —OC₁₋₄alkyl, —CF₃, —OCF₃, —CN, —NO₂, —S(O)C₁₋₄alkyl, —SO₂C₁₋₄alkyl, —CHO, —C(O)C₁₋₄alkyl, —CO₂C₁₋₄alkyl, —CO₂H, —C(O)NR^{d}R^{e}, or —NR^{d}R^{e};
      where R^{d} and R^{e} are each independently C₁₋₄alkyl;
   or two adjacent R^{c} groups taken together with the carbon members to which they are attached form a fused benzo ring; comprising
      intramolecularly cyclizing a compound of formula (II):

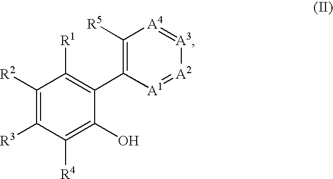

wherein R⁵ is chloro or bromo;
   in the presence of a copper(I) salt, in a polar, aprotic organic solvent.

The process for the preparation of compounds of Formula (I) may further comprise reacting a compound of formula (III):

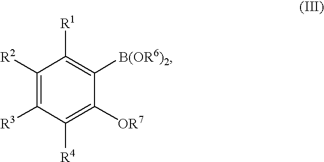

with a compound of formula (IV):

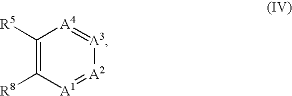

in the presence of a palladium(II) or palladium(0) catalyst and a ligand, and in the presence of a base, in a polar organic solvent;
to form a compound of formula (II);
wherein
R⁶ is H or C₁₋₄alkyl; or two R⁶ groups taken together form —C(CH₃)₂—C(CH₃)₂—;
R⁷ is H, C₁₋₄alkyl, methoxymethyl, (2-methoxyethoxy)methyl, benzyl, benzyloxymethyl, p-methoxybenzyl, trimethylsilyl, triethylsilyl, tert-butyldimethylsilyl, tert-butyldiphenylsilyl, or triisopropylsilyl; and
R⁸ is chloro, bromo, or iodo, when R⁵ is chloro, and R⁸ is bromo or iodo when R⁵ is bromo.

An object of the present invention is to overcome or ameliorate at least one of the disadvantages of the conventional methodologies and/or prior art, or to provide a useful alternative thereto.

Additional embodiments, features, and advantages of the invention will be apparent from the following detailed description and through practice of the invention.

### DETAILED DESCRIPTION OF INVENTION AND ITS PREFERRED EMBODIMENTS

For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by a / symbol), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

“Aryl”, also “Ar” or “aryl” or “arene”, includes phenyl, also “Ph”, and naphthyl, as well as the heteroaryl groups as defined below.

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

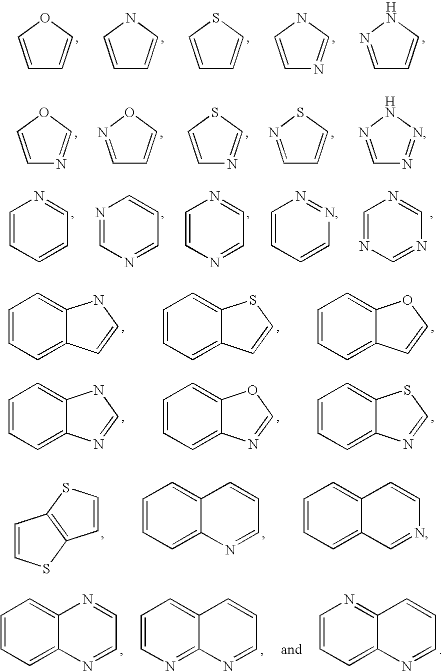

Those skilled in the art will recognize that the species of alkyl, aryl, and heteroaryl groups listed or illustrated above are not exhaustive, and that additional species within the scope of these defined terms may also be selected.

The term “halogen” represents chlorine, fluorine, bromine, or iodine. The term “halo” represents chloro, fluoro, bromo, or iodo.

The term “polar, aprotic organic solvent” refers to a solvent with a high dielectric constant (e.g. above 7.5), but which lacks hydroxyl groups or similar hydrogen-bond donating functionalities (Carey, F. A. and R. J. Sundberg, “Advanced Organic Chemistry,” 3^{rd} ed., 1990, Part B, p. 21). Examples of polar, aprotic organic solvents include, but are not limited to, tetrahydrofuran, N,N-dimethylformamide, N-methylpyrrolidone, acetone, N,N-dimethylsulfoxide, N,N-dimethylacetamide, and acetonitrile.

The term “polar organic solvent” refers to a solvent with a high dielectric constant (e.g. above 7.5). Polar organic solvents include polar, aprotic organic solvents (as described above) and polar, protic organic solvents that have a hydroxyl group or similar hydrogen-bonding functionality. In addition to the polar, aprotic organic solvents, examples of polar organic solvents include, but are not limited to methanol, ethanol, and the like.

The phrase “palladium(II) or palladium(0) catalyst and a ligand” includes conditions where the palladium species and the neutral ligand, such as a phosphine ligand, are added to the reaction mixture as separate reagents, or where the ligand(s) are pre-coordinated to the palladium species such that the palladium and ligand form a single reagent.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to represent hydrates, solvates, and polymorphs of such compounds, and mixtures thereof.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations given as percentages refer to mass ratios, unless indicated differently.

To provide a more concise description, examples of media such as solvents, reaction media and crystallization media are provided by a list of embodiments of such media without reciting explicitly that further embodiments are exemplified by chemically compatible mixtures of the explicitly recited embodiments. It is understood that, whether the terms “and chemically compatible mixtures thereof” or “and mixtures thereof” are recited explicitly or not, such examples are also considered illustrative examples in the list.

Reference to a chemical entity herein stands for a reference to any one of: (a) the actually recited form of such chemical entity, and (b) any of the forms of such chemical entity in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

Reference to a chemical entity herein by naming one of its forms stands for a reference to any one of: (a) the actually recited form of such chemical entity, and (b) any of the forms of such chemical entity in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, salvation, including hydration, protonation, and deprotonation. In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Inerest (ChEBI) dictionary of molecular entities. (See, for example its on line version at http://www.ebi.ac.uk/chebi/init.do). As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H ¹¹C, ¹³C, ¹⁴C, ¹⁵N, ¹⁸O, ¹⁷O, ³¹P, ³²P, 35S, ¹⁸F, ³⁶Cl, ¹²⁵I, respectively.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as R¹⁻⁸ and A¹⁻⁴, and any other generic substituent symbol used herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as R¹⁻⁸ and A¹⁻⁴, and any other generic substituent symbol used herein.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≦N≦m, with m>n.

Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

A “salt” is intended to mean a salt of a free acid or base of a compound represented by Formula (I). A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired alt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

List of Abbreviations

| | |
|---|---|
| dba | dibenzylideneacetone |
| Chx | cyclohexyl |
| dppf | 1,1′-bis(diphenylphosphino)ferrocene |
| MeOH | methanol |
| EtOH | ethanol |
| Ac | acetyl |
| DMF | N,N-dimethylformamide |
| DME | ethylene glycol dimethyl ether |
| THF | tetrahydrofuran |
| DMAc | N,N-dimethylacetamide |
| NMP | N-methylpyrrolidinone |

Providing a general synthesis, fused benzofuro-heterocycles of Formula (I) are prepared by a two-step sequence from readily available vicinal dihaloarenes of formula (IV) and optionally protected α-hydroxyboronic acid derivatives of formula (III) (Scheme 1). A halogen-selective Suzuki coupling between compounds (III) and (IV) affords biaryl compounds of formula (II) or (IIa). Where compounds of formula (IIa) are produced, a deprotection step provides compounds of formula (II). Compounds of formula (II) then undergo intramolecular cyclization to give benzofuro-heterocycles of Formula (I).

In some embodiments of Formula (I), R¹ is H; R² is H, chloro, or fluoro; R³ is H or fluoro; R⁴ is H; and each R^{c} is independently H, chloro, CF₃, CO₂H, or NO₂.

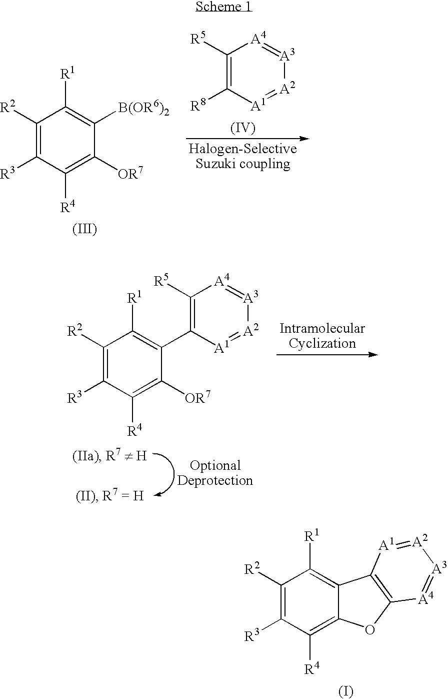

In initial studies in the context of this invention, Suzuki coupling between 2-chloroiodobenzene and 2-hydroxyphenylboronic acid indeed gave 2′-chloro-biphenyl-2-ol in high yield (Scheme 2). Generally, for embodiments of the invention where each of A¹⁻⁴ is CR^{c}, the Suzuki coupling occurred exclusively at the R⁸ position and no other coupling product was observed. Reactions are performed in the presence of a palladium(II) or palladium(0) catalyst and a ligand, and in the presence of a base, in a polar organic solvent. Suitable palladium(II) catalysts include, but are not limited to, Pd(OAc)₂, PdCl₂, and mixtures thereof. Suitable palladium(0) catalysts include Pd(PPh₃)₄, Pd₂(dba)₃, and mixtures thereof. Suitable ligands include, but are not limited to, phospine ligands and mixtures thereof. Examples of phosphine ligands include, but are not limited to, dppf, PPh₃, (tBu)₃P, and (Chx)₃P. In some embodiments, the molar amount of ligand used is twice the molar amount of palladium catalyst used. For example, in some embodiments, 5 molar % of palladium and 10 molar % of ligand are preferred. Suitable bases include, but are not limited to, K₃PO₄, KOH, K₂CO₃, Cs₂CO₃, Et₃N, NaOH, Na₃PO₄, Na₂CO₃, and mixtures thereof. Suitable polar organic solvents include, but are not limited to, acetonitrile, toluene, DMF, DME, THF, MeOH, EtOH, water, and mixtures thereof. Reactions are generally performed at temperatures from about room temperature to the reflux temperature of the solvent. In some embodiments, the palladium(II) catalyst is Pd(OAc)₂, the base is K₃PO₄, and the polar organic solvent is acetonitrile. In some embodiments, the reaction is performed at a temperature that is about room temperature.

In further studies in the context of this invention, Ullmann-type intramolecular cyclization of 2′-chloro-biphenyl-2-ol provided dibenzofuran.

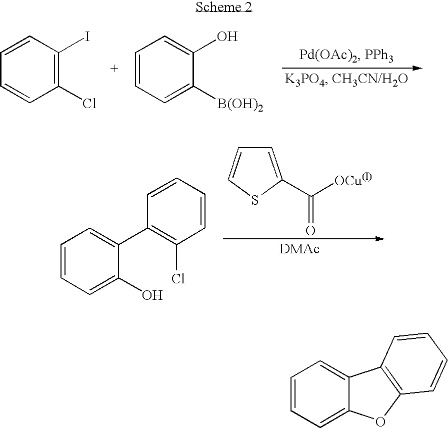

Generally, the intramolecular cyclization reaction is promoted by copper(I) salts, such as copper(I) thiophene-2-carboxylate (CuTC), CuCl, CuBr, CuOAc, or a mixture thereof, in a polar, aprotic organic solvent such as DMAc, NMP, DMF, or a mixture thereof. In some embodiments, at least one molar equivalent of the copper(I) salt is employed, and in other embodiments, from about 1.1 to about 1.3 molar equivalents of the copper(I) salt are employed. In some embodiments, CuTC¹² is employed as the promoter. In some embodiments, the solvent is DMAc or NMP. In some embodiments, the cyclization reactions are performed at a temperature from about room temperature to about 140° C. Heating is accomplished using traditional heating methods or microwave irradiation. Preferably, for embodiments where each of A¹⁻⁴ is CR^{c}, the cyclization is performed at a temperature from about 80° C. to about 140° C., and more preferably from about 120° C. to about 140° C. The overall two-step route proved applicable to the synthesis of various dibenzofurans (where each of A¹⁻⁴ is CR^{c}) from 1,2-dihalobenzenes and 2-hydroxyphenylboronic acid derivatives (Table 1, entries 1-3).

In some experiments, unprotected 2-hydroxyphenylboronic acid derivatives were used as the starting material (compounds of formula (III) where R⁷ is H). However, only a handful of these reagents are commercially available. On the other hand, O-protected α-hydroxyphenylboronic acid derivatives (compounds of formula (III) where R⁷ is not H), in particular α-methoxyphenylboronic acid derivatives (where R⁷ is methyl), are widely available and often much less expensive. For example, 5-chloro-2-methoxyphenylboronic acid is currently inexpensive, but its unprotected counterpart, 5-chloro-2-hydroxyphenylboronic acid, currently costs nearly 30 times as much from the same vendor (Combi-blocks). Thus, halogen-selective Suzuki reaction of compounds of formula (III), where R⁷ is not H, with compounds of formula (IV) provides compounds of formula (IIa) (Scheme 1, supra). Compounds of formula (IIa) are converted to compounds of formula (II) using deprotection methods known in the art. In a particular embodiment (Table 1, entry 4), α-methoxyphenylboronic acids undergo halogen-selective Suzuki coupling (using conditions as described for Scheme 1). Subsequent demethylation in the presence of BBr₃, in a solvent such as CH₂Cl₂, affords compounds of formula (II) in good yield.¹⁴ Compounds of formula (II) then undergo intramolecular cyclization using conditions as described in Scheme 1 to give the corresponding dibenzofuran products.

TABLE 1
Preparation of dibenzofuran derivatives.

| Entry | Formula (IV) | Formula (III) | Formula (II) (yield)^{a} | Formula (I) (yield)^{a} |
|---|---|---|---|---|
| 1 | 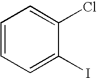 | 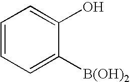 | 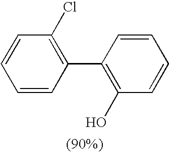 | 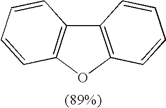 |
| 2 | 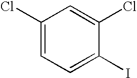 | 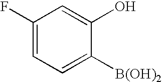 | 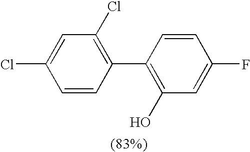 | 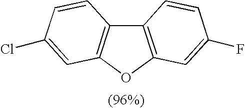 |
| 3 | 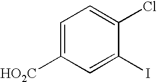 | 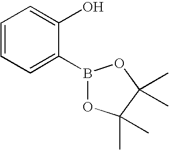 | 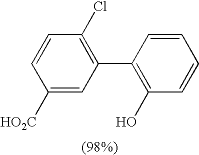 | 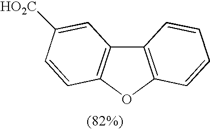 |
| 4 | 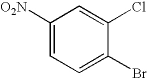 | 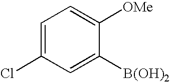 | 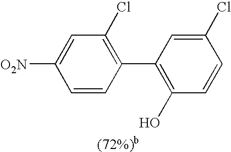 | 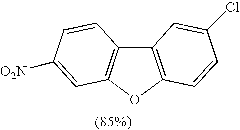 |

^{a}Isolated yield;
^{b}Isolated yield after halogen-selective Suzuki coupling and deprotection steps.

The methodology of the present invention is applicable to the synthesis of additional embodiments of Formula (I), where at least one of A¹⁻⁴ is N, including benzofuropyridines, benzofuropyrazines, and benzofuropyrimidines (Table 2). As described above, the first step is a halogen-selective Suzuki coupling reaction.¹⁵ Good yields and regioselectivity were observed.¹⁶ Notably, all four benzofuropyridine regioisomers were readily prepared from the appropriate dihalopyridines in two or three steps and high overall yield (entries 2-5).

One skilled in the art will recognize that for embodiments where the A¹⁻⁴-containing ring is a pyridine or pyrimidine ring, and where R⁵ and R⁸ are both chloro or both bromo, the different reactivity of halogen atoms determines regioselectivity of the Suzuki coupling. For example, with a pyridine ring, a halogen at the 2-position (“ortho” to the nitrogen ring member) reacts selectively over the same halogen at the 4-position (“para” to the nitrogen ring member), which in turn reacts selectively over the same halogen at the 3-position (“meta” to the nitrogen ring member) (entries 3, 5, and 8). As a further example, for a pyrimidine ring, a halogen at the 4-position reacts selectively over the same halogen at the 2-position, which in turn reacts selectively over the same halogen at the 5-position (entry 8). Additionally, where R⁵ and R⁸ are both chloro or bromo, and were bound at chemically identical positions (e.g. symmetrical positions), mono-coupling was achieved when a stoichiometric amount or slight excess of boronic acid derivative of formula (III) was used (entry 7).

The second key step, the Cu(I)-promoted Ullmann-type intramolecular cyclization,¹⁷ is also applicable to embodiments of Formula (I) where at least one of A¹⁻⁴ is N. Generally, the intramolecular cyclization reaction is promoted by copper(I) salts, such as copper(I) thiophene-2-carboxylate (CuTC), CuCl, CuBr, CuOAc, or a mixture thereof, in a polar, aprotic organic solvent such as DMAc, NMP, DMF, or a mixture thereof. In some embodiments, at least one molar equivalent of the copper(I) salt is employed, and in other embodiments, from about 1.1 to about 1.3 molar equivalents of the copper(I) salt are employed. In some embodiments, CuTC is employed as the promoter. In some embodiments, the solvent is DMAc or NMP. In some embodiments, the cyclization reactions are performed at a temperature from about room temperature to about 140° C. Heating is accomplished using traditional heating methods or microwave irradiation. Preferably, for embodiments where at least one of A¹⁻⁴ is N, the reaction temperature is preferably from about 40° C. to about 140° C., and more preferably from about 60° C. to 100° C.

TABLE 2
Preparation of benzofuro-heterocycles.

| | |
|---|---|
| 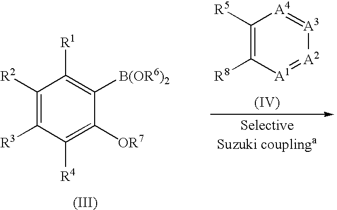 | 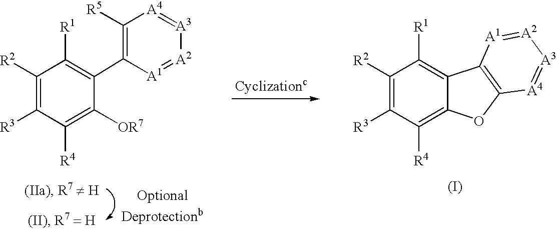 |

| Entry | Formula (IV) | Formula (III) | Formula (II) (yield)^{d} | Formula (I) (yield)^{d} |
|---|---|---|---|---|
| 1 | 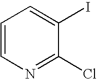 | 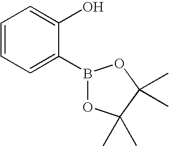 | 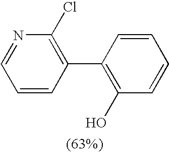 | 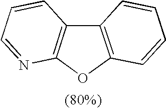 |
| 2 | 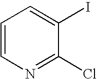 | 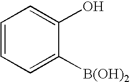 | 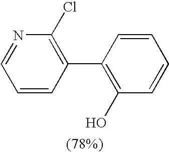 | 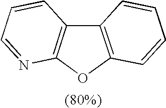 |
| 3 | 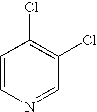 | 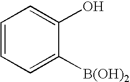 | 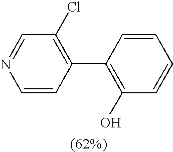 | 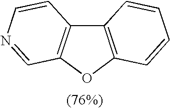 |
| 4 | 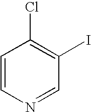 | 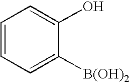 | 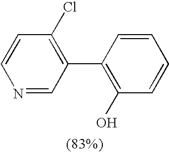 | 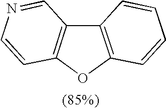 |
| 5 | 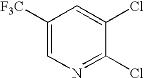 | 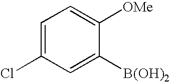 | 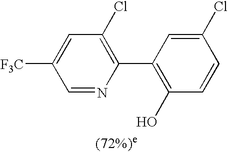 | 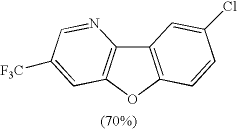 |
| 6 | 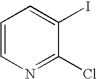 | 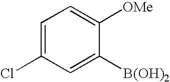 | 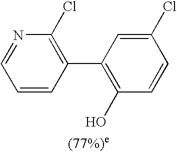 | 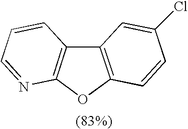 |
| 7 | 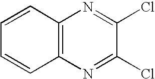 | 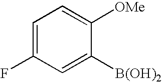 | 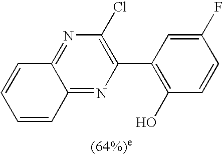 | 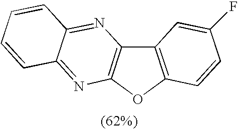 |
| 8 | 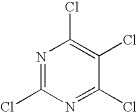 | 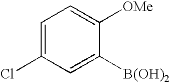 | 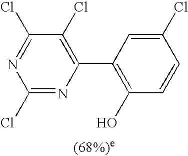 | 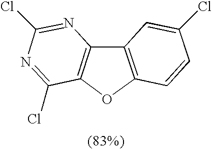 |

^{a}Pd(OAc)₂, PPh₃, K₃PO₄, CH₃CN, H₂O, rt to 80° C.;
^{b}BBr₃, CH₂Cl₂, rt;
^{c}Cu(I) thiophene-2-carboxylate (CuTC), DMAc or NMP, 70° C. to 100° C.;
^{d}Isolated yield;
^{e}Isolated yield after halogen-selective Suzuki coupling and deprotection steps.

Compared to traditional Ullmann cyclization methods, the intramolecular cyclization reaction conditions of the present invention are generally much milder and often result in higher yields (Table 3, entry 2 vs. 1; 4 vs. 3; 6 vs. 5; and 8 vs. 7). In addition, most Ullmann coupling reactions in the literature require stoichiometric or excess base and therefore are not compatible with base-sensitive substrates. The present invention involves neutral conditions, and base-sensitive compounds are well tolerated. For example, 2-(2′,5′,6′-trichloropyrimidyl)phenol, which decomposes quickly under basic conditions even at room temperature (Table 3, entry 9), is an excellent substrate under the reaction conditions of the present invention (entry 11).

TABLE 3
Cu(I)-promoted intramolecular cyclization vs. traditional conditions.

| Entry | Formula (II) | Formula (I) | Conditions | Yield |
|---|---|---|---|---|
| 1 | 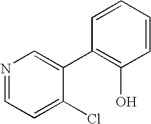 | 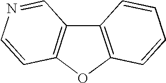 | NaO^{t}Bu, DMSOreflux | 34%^{(Ref 7)} |
| 2 | ″ | ″ | CuTC, DMAc | 85% |
| | | | 100° C., 5 h | |
| 3 | 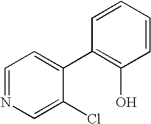 | 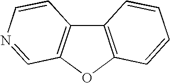 | NaO^{t}Bu, DMSO190° C. | 57%^{(Ref 7)} |
| 4 | ″ | ″ | CuTC, DMAc | 76% |
| | | | 100° C., 5 h | |
| 5 | 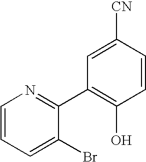 | 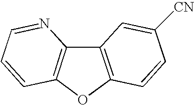 | 1) KOH, 100° C., 3 h;2) Cu, 200° C., 12 h | 70%^{(Ref 6)} |
| 6 | 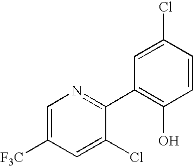 | 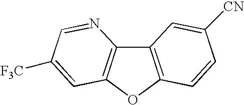 | CuTC, DMAc80° C., 1 h | 70% |
| 7 | 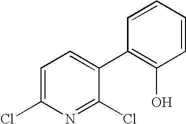 | 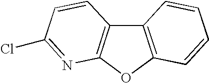 | K₂CO₃, CH₃CN82° C., 5 h | 70%^{(Ref 10)} |
| 8 | 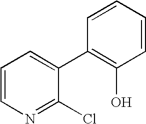 | 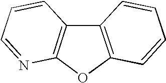 | CuTC, DMAc70° C., 6 h | 80% |
| 9 | 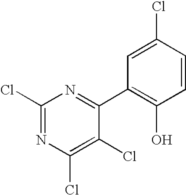 | 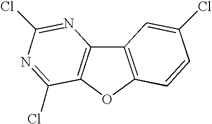 | K₂CO₃, acetonert, 1.5 h | decomp. |
| 10 | ″ | ″ | Toluene | no reaction |
| | | | 170° C., 1 h ^{a} | |
| 11 | ″ | ″ | CuTC, DMAc | 76% |
| | | | 80° C., 1 h | |

^{a} Under microwave irradiation.

Finally, all starting materials listed in Tables 1 and 2 were purchased from commercial vendors and used directly. Since many α-hydroxyphenylboronic acid derivatives (formula (III)) and 1,2-dihaloarenes (formula (IV)) are commercially available, a wide variety of dibenzofurans, benzofuropyridines, benzofuropyrazines, and benzofuropyrimidines are readily synthesized in two or three steps via this route. The generality of the reaction conditions for both the halogen-selective Suzuki coupling and intramolecular cyclization reactions makes the method suitable for library preparations.

With respect to the above schemes, artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables in the above schemes are as defined in reference to Formula (I).

The present invention provides a concise synthesis for the facile assembly of fused benzofuro-heterocycles, which are important structural motifs in biologically active compounds and drug candidates. The key reactions are halogen-selective Suzuki coupling and Cu(I)-mediated intramolecular Ullmann-type cyclization under neutral and relatively mild conditions. This route has broad substrate scope and should be applicable for the preparation of many pharmacologically interesting compounds.

The following specific examples are provided to further illustrate the invention and various preferred embodiments.

### EXAMPLES

### Chemistry Methods:

In obtaining the compounds described in the examples below and the corresponding analytical data, the following experimental and analytical protocols were followed unless otherwise indicated.

Reagents were purchased from commercial suppliers and were used without purification unless otherwise noted. N,N-Dimethylacetamide (DMAc) was dried via passage through two alumina columns according to the procedure of Grubbs (Pangborn, A. B.; Giardello, M. A.; Grubbs, R. H.; Rosen, R. K.; Timmers, F. J. A Safe and Convenient Procedure for Solvent Purification. Organometallics 1996, 15, 1518-1520). N-Methylpyrrolidone (NMP) was dried over activated 4 Å molecular sieves overnight.

Unless otherwise stated, reaction mixtures were magnetically stirred at room temperature (rt). Where mixtures, solutions, and extracts were “concentrated”, they were typically concentrated on a rotary evaporator under reduced pressure. Microwave irradiation was carried out on a CEM Explorer instrument (CEM Corp., Matthews, N.C. 28106). Flash column chromatography was performed on CombiFlash Companion systems (CombiFlash Inc.) using pre-packed ISCO Redisep cartridges. Where regioisomeric mixtures are obtained, single isomers may be separated using conventional methods such as chromatography or crystallization.

Compounds of Formula (I) may be converted to their corresponding salts using methods described in the art. For example, an amine of Formula (I) is treated with trifluoroacetic acid, HCl, or citric acid in a solvent such as Et₂O, CH₂Cl₂, THF, or MeOH to provide the corresponding salt form.

Nuclear magnetic resonance (NMR) spectra were obtained on Bruker model DRX spectrometers. Chemical shifts (δ) are reported in parts per million downfield from an internal tetramethylsilane standard. Spin multiplets are given as s (singlet), d (doublet), t (triplet), q (quartet), or m (multiplet). Coupling constants (J) are given in hertz (Hz). Mass spectra were recorded on a Hewlett-Packard 1100MSD using electrospray ionization (ESI), or a Hewlett-Packard 5973MSD using electron impact ionization (EI) in either positive or negative mode as indicated. High resolution mass spectrometry (HRMS) (ESI) was performed on a Bruker μTof.

Chemical names were generated using ChemDraw Version 6.0.2 (CambridgeSoft, Cambridge, Mass.) or ACD/Name Version 9 (Advanced Chemistry Development, Toronto, Ontario, Canada).

### Representative Procedure A: Halogen-selective Suzuki coupling with 2-O-unprotected 2-hydroxyphenyl boronic acid

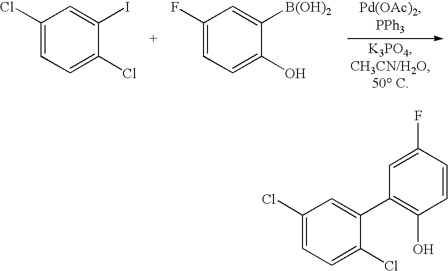

2′,5′-Dichloro-5-fluoro-biphenyl-2-ol. In a 20 mL vial, acetonitrile (4 mL) and water (1 mL) were sparged with nitrogen for 1 minute. Potassium phosphate (0.42 g, 1.97 mmol, 2.0 equiv), triphenylphosphine (26 mg, 0.098 mmol, 0.1 equiv), 1,4-dichloro-2-iodobenzene (0.133 mL, 0.98 mmol, 1.0 equiv), 5-fluoro-2-hydroxy-phenylboronic acid (0.23 g, 1.48 mmol, 1.5 equiv), and palladium acetate (11 mg, 0.049 mmol, 0.05 equiv) were added sequentially. The reaction was sealed under a nitrogen atmosphere and heated to 50° C. for 20 h. After removal of acetonitrile solvent by rotary evaporation, the reaction mixture was diluted with water (10 mL) and extracted with CH₂Cl₂ (10 mL). The organic layer was washed with water (10 mL) and saturated (satd.) aqueous (aq.) NaCl (10 mL), dried over Na₂SO₄, and concentrated. The crude product was purified by silica gel column chromatography (eluent: 0-70% CH₂Cl₂ in hexanes). The product was obtained as a colorless oil (210 mg, 83%). ¹H NMR (600 MHz, CDCl₃, δ): 7.44 (d, J=9.3 Hz, 1H), 7.34-32 (m, 2H), 7.10 (dd, J=8.4, 6.4 Hz, 1H), 6.74-6.70 (m, 2H), 4.97 (br s, 1H); ¹³C NMR (150 MHz, CDCl₃, δ): 163.6 (d, J=247.4 Hz), 153.7 (d, J=12.0 Hz), 136.5, 133.1, 132.5, 132.1, 131.6 (d, J=10.1 Hz), 131.2, 129.8, 121.0 (d, J=3.3 Hz), 107.9 (d, J=21.7 Hz), 103.7 (d, J=24.9 Hz); MS (EI+): calculated for C₁₂H₇Cl₂FO, 256.0; m/z found, 255.9 [M^{·+}].

### Representative Procedure B. Copper(I) thiophene-2-carboxylate (CuTC)-mediated intramolecular cyclization (with acidic workup)

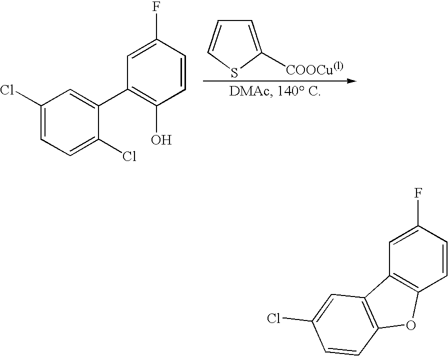

2-Chloro-8-fluoro-dibenzofuran. A 10 mL microwave vial was charged with 2′,5′-dichloro-5-fluoro-biphenyl-2-ol (50 mg, 0.19 mmol, 1.0 eq) and DMAc (2.0 mL). The solution was sparged with dry nitrogen for 2 min. Copper(I) thiophene-2-carboxylate (CuTC, 48 mg, 0.25 mmol, 1.3 equiv) was then added. The vial was sealed under a nitrogen atmosphere and heated under microwave irradiation at 140 C. for 20 min. The reaction mixture was diluted with 0.2 M HCl (10 mL) and extracted with CH₂Cl₂ (10 mL). The organic layer was washed with satd. aq. NaHCO₃ (10 mL) and water (10 mL×3), dried over Na₂SO₄, and concentrated. The crude product was purified by silica gel column chromatography (eluent: 20-70% CH₂Cl₂ in hexanes). The product was obtained as a white solid (41 mg, 96%). ¹H NMR (600 MHz, CDCl₃, δ): 7.83 (d, J=2.1 Hz, 1H), 7.81 (dd, J=8.6, 5.4 Hz, 1H), 7.45 (d, J=8.7 Hz, 1H), 7.37 (dd, J=8.7, 2.2 Hz, 1H), 7.26 (dd, J=8.8, 2.2 Hz, 1H), 7.09 (td, J=9.0, 2.3 Hz, 1H); ¹³C NMR (150 MHz, CDCl₃, δ): 162.7 (d, J=246.5 Hz), 157.1 (d, J=13.5 Hz), 155.1 (d, J=2.2 Hz), 128.6, 126.7, 125.1, 121.4 (d, J=10.4 Hz), 120.8, 119.6 (d, J=2.1 Hz), 112.6, 111.2 (d, J=24.0 Hz), 99.9 (d, J=26.9 Hz); MS (EI+): calculated for C₁₂H₆ClFO, 220.0; m/z found, 220.1 [M^{·+}].

### Representative Procedure C. Copper(I) thiophene-2-carboxylate (CuTC)-mediated intramolecular cyclization (with basic workup)

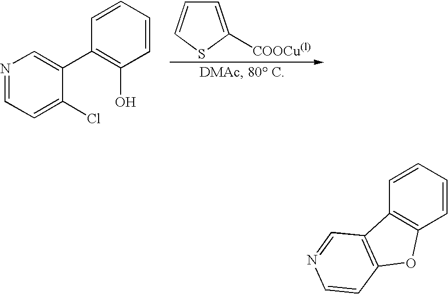

Benzo[4,5]furo[3,2-c]pyridine. A 10 mL flask was charged with 2-(4-chloro-pyridin-3-yl)-phenol (0.20 g, 0.97 mmol, 1.0 eq) and DMAC (4.0 mL). The solution was sparged with dry nitrogen for 2 min. CuTC (0.24 g, 1.26 mmol, 1.3 equiv) was then added. The reaction was heated at 80° C. for 6 h under a nitrogen atmosphere. The reaction mixture was diluted with aq. 0.5 M ethylenediamine tetraacetic acid (EDTA) (16 mL). The pH was adjusted to ˜11 with 1 M NaOH solution. The mixture was then extracted with CH₂Cl₂ (20 mL). The organic layer was washed with water (20 mL×3) and satd. aq. NaCl (20 mL), dried over Na₂SO₄, and concentrated. The crude product was purified by silica gel column chromatography (eluent: 0-20% ethyl acetate in CH₂Cl₂). The product was obtained as a white solid (0.14 mg, 85%). ¹H NMR (600 MHz, CDCl₃, δ): 9.26 (s, 1H), 8.65 (d, J=5.7 Hz, 1H), 8.02 (d, J=7.7 Hz, 1H), 7.62 (d, J=8.3 Hz, 1H), 7.53 (td, J=7.3, 1.3 Hz, 1H), 7.51 (dd, J=5.7, 0.9 Hz, 1H), 7.42 (td, J=7.6, 0.9 Hz, 1H); ¹³C NMR (150 MHz, CDCl₃, δ): 160.9, 155.9, 147.4, 143.6, 128.3, 123.9, 121.6, 121.5, 121.1, 112.0, 107.5; HRMS (ESI+): calculated for C₁₁H₈NO [M+H⁺], 170.0600; m/z found, 170.0594.

### Representative Procedure D. Halogen-selective Suzuki coupling with 2-O-protected 2-hydroxyphenyl boronic acid

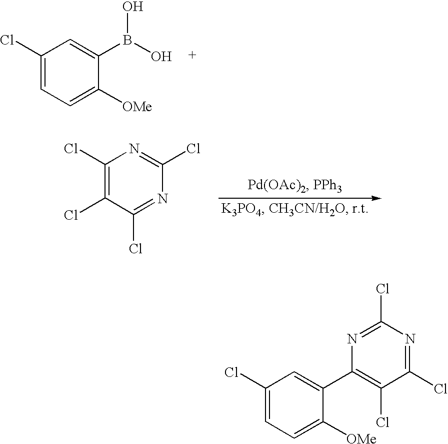

2,4,5-Trichloro-6-(5-chloro-2-methoxy-phenyl)-pyrimidine. In a 250 mL three-neck flask fitted with a degassing tube and temperature probe, acetonitrile (100 mL) and water (25 mL) were degassed with nitrogen for 30 min while stirring. 2,4,5,6-Tetrachloropyrimidine (8.77 g, 0.0402 mol, 1.5 equiv) and triphenylphosphine (0.70 g, 2.6 mmol, 0.1 equiv) were added and degassed for 15 min. 5-Chloro-2-methoxyphenylboronic acid (5.00 g, 0.0268 mol, 1.0 equiv), potassium phosphate (11.39 g, 0.0536 mol, 2.0 equiv) and palladium acetate (301 mg, 1.3 mmol, 0.05 equiv) were added and degassed for 5 min. The reaction was complete after 2 h at room temperature. The reaction mixture was added to 250 mL CH₂Cl₂. The organic layer was washed twice with water (125 mL), dried over Na₂SO₄, and concentrated. The crude product was purified by silica gel column chromatography. The product was obtained as a white solid (5.97 g, 69%). ¹H NMR (500 MHz, CDCl₃, δ): 7.45 (dd, J=8.9, 2.6 Hz, 1H), 7.31 (d, J=2.6 Hz, 1H), 6.94 (d, J=8.9 Hz, 1H), 3.82 (s, 3H); MS (ESI+): calculated for C₁₁H₆Cl₄N₂O, 321.92; m/z found, 323.0 [M+H⁺].

### Representative Procedure E. Demethylation of biaryl anisole

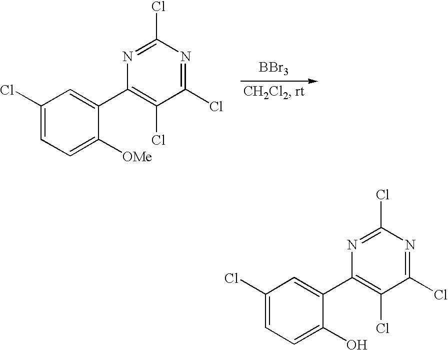

4-Chloro-2-(2,5,6-trichloro-pyrimidin-4-yl)-phenol. A 250 mL 2-neck flask fitted with a rubber septum, temperature probe, and addition funnel was charged with 2,4,5-trichloro-6-(5-chloro-2-methoxy-phenyl)-pyrimidine (5.97 g, 18.54 mmol) and CH₂Cl₂ (90 mL) and kept under a nitrogen atmosphere. 1 M BBr₃ in CH₂Cl₂ (37.1 mL, 37.1 mmol, 2 equiv.) was added slowly via addition funnel so that the temperature never exceeded 25° C. The reaction was complete 1 h after complete addition of BBr₃. Water (90 mL) was added and the reaction stirred for approximately 30 min. The organic layer was washed with satd. aq. NaHCO₃, dried over Na₂SO₄, and concentrated. The product was obtained as a yellow powder (5.6 g, 98%). ¹H NMR (500 MHz, CDCl₃, δ): 9.76 (s, 1H), 8.00 (d, J=2.6 Hz, 1H), 7.39 (dd, J=8.9, 2.6 Hz, 1H), 7.03 (d, J=8.9 Hz, 1H); MS (ESI+): calculated for C₁₀H₄Cl₄N₂O, 307.91; m/z found, 308.9 [M+H]⁺.

### 2′-Chloro-biphenyl-2-ol

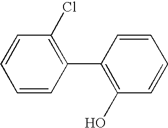

The title compound was prepared using methods analogous to those in Representative Procedure A. ¹H NMR (600 MHz, CDCl₃, δ): 7.54-7.51 (m, 1H), 7.37-7.34 (m, 3H), 7.31 (tdd, J=8.2, 1.6, 0.8 Hz, 1H), 7.17 (ddd, J=7.4, 1.3, 0.6 Hz, 1H), 7.02-6.97 (m, 2H), 4.98 (s, 1H); ¹³C NMR (150 MHz, CDCl₃, δ): 152.6, 135.7, 134.1, 132.1, 130.7, 130.1, 129.8, 129.6, 127.3, 125.9, 120.5, 115.8; MS (EI+): calculated for C₁₂H₉ClO [M^{·+}], 204.0; m/z found, 204.1.

### Dibenzofuran

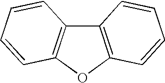

The title compound was prepared using method analogous to those in Representative Procedure B. Spectral data was identical to obtained for commercial material.

### 6-Chloro-2′-hydroxy-biphenyl-3-carboxylic acid

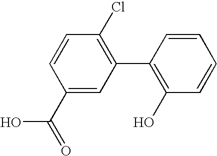

The title compound was prepared using methods analogous to those in Representative Procedure A. ¹H NMR (600 MHz, CDCl₃, δ): 13.2 (br s, 1H), 9.60 (s, 1H), 7.89 (dd, J=8.3, 2.1 Hz, 1H), 7.81 (d, J=2.1 Hz, 1H), 7.65 (d, J=8.3 Hz, 1H), 7.24 (ddd, J=9.1, 7.5, 1.7 Hz, 1H), 7.12 (dd, J=7.5, 1.7 Hz, 1H), 6.95 (dd, J=8.2, 0.9 Hz, 1H), 6.88 (td, J=7.4, 1.1 Hz, 1H);

### Dibenzofuran-2-carboxylic acid

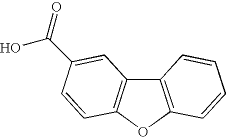

The title compound was prepared using methods analogous to those in Representative Procedure B. ¹H NMR (500 MHz, d₆-DMSO, δ): 13.00 (br s, 1H), 8.78 (s, 1H), 8.30 (d, J=7.5 Hz, 1H), 8.13 (d, J=8.7 Hz, 1H), 7.80 (d, J=8.6 Hz, 1H), 7.75 (d, J=8.3 Hz, 1H), 7.58 (td, J=7.2, 1.2 Hz, 1H), 7.45 (t, J=7.3 Hz, 1H); ¹³C NMR (126 MHz, d₆-DMSO, δ): 167.1, 157.9, 156.0, 129.0, 128.2, 125.9, 123.8, 123.5, 123.0, 123.0, 121.7, 111.8, 111.6.

### 5,2′-Dichloro-4′-nitro-biphenyl-2-ol

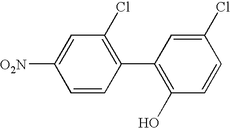

The title compound was prepared using methods analogous to those in Representative Procedures D and E. ¹H NMR (500 MHz, CDCl₃, δ): 8.40 (d, J=2.3 Hz, 1H), 8.22 (dd, J=8.4, 2.3 Hz, 1H), 7.55 (d, J=8.4 Hz, 1H), 7.33 (dd, J=8.7, 2.6 Hz, 1H), 7.19 (d, J=2.6 Hz, 1H), 6.93 (d, J=8.7 Hz, 1H), 4.9(s, 1H); ¹³C NMR (126 MHz, CDCl₃, δ): 151.1, 148.1, 142.1, 135.1, 132.6, 130.4, 130.1, 125.9, 125.9, 125.1, 121.9, 117.6; MS (EI+): calculated for C₁₂H₇Cl₂NO₃, 283.0; m/z found, 282.8 [M^{·+}].

### 2-Chloro-7-nitro-dibenzofuran

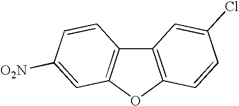

The title compound was prepared using methods analogous to those in Representative Procedure B. ¹H NMR (500 MHz, CDCl₃, δ): 8.48 (d, J=1.9 Hz, 1H), 8.32 (dd, J=8.5, 1.9 Hz, 1H), 8.05 (d, J=8.5 Hz, 1H), 8.03 (d, J=2.0 Hz, 1H), 7.61 (d, J=8.8 Hz, 1H), 7.57 (dd, J=8.8, 2.0 Hz, 1H); ¹³C NMR (126 MHz, CDCl₃, δ): 156.6, 155.7, 129.6, 129.5, 129.1, 123.9, 121.5, 120.8, 118.7, 113.4, 108.2; MS (EI+): calculated for C₁₂H₆ClNO₃, 247.0; m/z found, 246.8 [M^{·+}].

### 2-(2-Chloro-pyridin-3-yl)-phenol

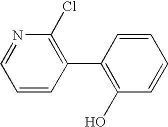

The title compound was prepared using methods analogous to those in Representative Procedure A. ¹H NMR (500 MHz, d₆-DMSO, δ): 9.67 (s, 1H), 8.39 (dd, J=4.7, 1.9 Hz, 1H), 7.78 (dd, J=9.4, 1.9 Hz, 1H), 7.46 (dd, J=7.4, 4.7 Hz, 1H), 7.25 (td, J=9.4, 1.4 Hz, 1H), 7.13 (dd, J=7.5, 1.5 Hz, 1H), 6.94 (d, J=8.1 Hz, 1H), 6.88 (t, J=7.4 Hz, 1H); ¹³C NMR (126 MHz, d₆-DMSO, δ): 154.3, 149.8, 148.2, 140.8, 134.0, 130.5, 129.6, 124.2, 122.8, 118.7, 115.5; HRMS (ESI+): calculated for C₁₁H₉ClNO [M+H⁺], 206.0367; m/z found, 206.0366.

### Benzo[4,5]furo[2,3-b]pyridine

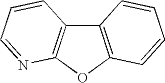

The title compound was prepared using methods analogous to those in Representative Procedure C. ¹H NMR (500 MHz, CDCl₃, δ): 8.45 (dd, J=4.9, 1.7 Hz, 1H), 8.25 (dd, J=7.6, 1.7 Hz, 1H), 7.94 (pseudo d, J=8.4 Hz, 1H), 7.64 (pseudo d, J=8.3 Hz, 1H), 7.52 (td, J=7.4, 1.3 Hz, 1H), 7.38 (td, J=7.6, 0.9 Hz, 1H), 7.33 (dd, J=7.6, 4.9 Hz, 1H); ¹³C NMR (126 MHz, CDCl₃, δ): 163.2, 154.5, 146.4, 129.6, 128.3, 123.3, 122.5, 121.2, 119.1, 116.9, 112.1; HRMS (ESI+): calculated for C₁₁H₈NO [M+H⁺], 170.0600; m/z found, 170.0618.

### 2-(3-Chloro-pyridin-4-yl)-phenol

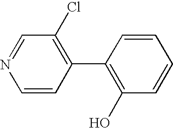

The title compound was prepared using methods analogous to those in Representative Procedure A. ¹H NMR (600 MHz, d₆-DMSO, δ): 9.91 (s, 1H), 8.68 (s, 1H), 8.52 (d, J=4.8 Hz, 1H), 7.39 (d, J=4.9 Hz, 1H), 7.27 (td, J=8.3, 1.2 Hz, 1H), 7.14 (dd, J=7.5, 1.4 Hz, 1H), 6.96 (d, J=8.2 Hz, 1H), 6.89 (t, J=7.4 Hz, 1H); ¹³C NMR (150 MHz, d₆-DMSO, δ): 154.4, 148.7, 147.5, 145.8, 130.8, 130.1, 130.0, 126.5, 123.3, 118.6, 115.7; HRMS (ESI+): calculated for C₁₁H₉ClNO [M+H⁺], 206.0367; m/z found, 206.0363.

### Benzo[4,5]furo[2,3-c]pyridine

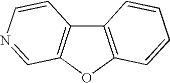

The title compound was prepared using methods analogous to those in Representative Procedure C. ¹H NMR (600 MHz, CDCl₃, δ): 9.00 (s, 1H), 8.60 (d, J=5.1 Hz, 1H), 8.02 (d, J=7.9 Hz, 1H), 7.87 (dd, J=5.1, 0.9 Hz, 1H), 7.64 (m, 1H), 7.42 (td, J=8.0, 1.3 Hz, 1H), 7.38 (td, J=7.6, 0.9 Hz, 1H); ¹³C NMR (150 MHz, CDCl₃, δ): 156.8, 152.8, 143.0, 134.4, 130.9, 129.9, 123.4, 122.2, 122.1, 115.1, 112.5; HRMS (ESI+): calculated for C₁₁H₈NO [M+H⁺], 170.0600; m/z found, 170.0613.

### 2-(4-Chloro-pyridin-3-yl)-phenol

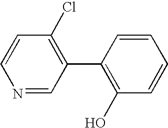

The title compound was prepared using methods analogous to those in Representative Procedure A. ¹H NMR (600 MHz, d₆-DMSO, δ): 9.74 (br s, 1H), 8.50 (d, J=5.3 Hz, 1H), 8.47 (s, 1H), 7.62 (d, J=5.4 Hz, 1H), 7.27 (td, J=9.1, 1.7 Hz, 1H), 7.15 (dd, J=7.5, 1.6 Hz, 1H), 6.96 (dd, J=8.1, 0.7 Hz, 1H), 6.90 (td, J=7.4, 1.0 Hz, 1H); ¹³C NMR (150 MHz, d₆-DMSO, δ): 154.8, 151.6, 149.1, 142.4, 133.7, 130.7, 129.9, 124.2, 122.2, 118.8, 115.5; HRMS (ESI+): calculated for C₁₁H₉ClNO [M+H⁺], 206.0367; m/z found, 206.0362.

### 4-Chloro-2-(3-chloro-5-trifluoromethyl-pyridin-2-yl)-phenol

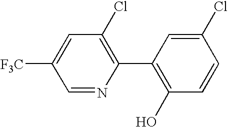

The title compound was prepared using methods analogous to those in Representative Procedures D and E. ¹H NMR (500 MHz, CDCl₃, δ): 11.0 (s, 1H), 8.78 (d, J=0.9 Hz, 1H), 8.17 (d, J=1.6 Hz, 1H), 8.10 (d, J=2.6 Hz, 1H), 7.35 (dd, J=8.8, 2.6 Hz, 1H), 7.04 (d, J=8.8 Hz, 1H); ¹³C NMR (126 MHz, CDCl₃, δ): 156.5, 156.3, 142.2 (q, J=3.9 Hz), 137.7 (q, J=3.5 Hz), 132.5, 129.8, 129.6, 126.1 (q, J=34 Hz), 123.7, 122.2 (q, J=273 Hz), 119.9, 119.7; HRMS (ESI+): calculated for C₁₂H₇Cl₂F₃NO [M+H⁺], 307.9851; m/z found, 307.9853.

### 8-Chloro-3-trifluoromethyl-benzo[4,5]furo[3,2-b]pyridine

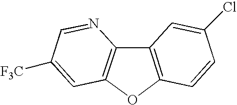

The title compound was prepared using methods analogous to those in Representative Procedure B. ¹H NMR (500 MHz, CDCl₃, δ): 8.95 (s, 1H), 8.27 (s, 1H), 8.10 (s, 1H), 7.65-7.61 (m, 2H); ¹³C NMR (126 MHz, CDCl₃, δ): 156.9, 149.1, 146.5, 142.6 (q, J=4.1 Hz), 130.8, 130.1, 124.8 (q, J=27.6 Hz), 123.7, 123.6 (q, J=272.8 Hz), 121.7, 116.3 (q, J=3.8 Hz), 113.7; HRMS (ESI+): calculated for C₁₂H₆ClF₃NO [M+H⁺], 272.0085; m/z found, 272.0077.

### 4-Chloro-2-(2-chloro-pyridin-3-yl)-phenol

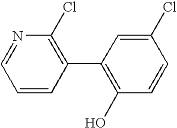

The title compound was prepared using methods analogous to those in Representative Procedure A. ¹H NMR (500 MHz, CDCl₃, δ): 8.41 (dd, J=4.8, 1.9 Hz, 1H), 7.73 (dd, J=7.5, 1.9 Hz, 1H), 7.35 (dd, J=7.5, 4.8 Hz, 1H), 7.29 (dd, J=8.7, 2.6 Hz, 1H), 7.19 (d, J=2.6 Hz, 1H), 6.95 (d, J=8.7 Hz, 1H), 6.05 (br s, 1H); ¹³C NMR (126 MHz, CDCl₃, δ): 157.8, 150.7, 149.1, 140.9, 132.4, 130.4, 130.1, 125.8, 125.74, 122.6, 117.7; HRMS (ESI+): calculated for C₁₁H₈Cl₂NO [M+H⁺], 239.9977; m/z found, 239.9976.

### 6-Chloro-benzo[4,5]furo[2,3-b]pyridine

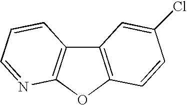

The title compound was prepared using methods analogous to those in Representative Procedure B. ¹H NMR (500 MHz, CDCl₃, δ): 8.50 (dd, J=4.9, 1.7 Hz, 1H), 8.25 (dd, J=7.6, 1.7 Hz, 1H), 7.93 (d, J=2.2 Hz, 1H), 7.59 (d, J=8.7 Hz, 1H), 7.50 (dd, J=8.7, 2.2 Hz, 1H), 7.37 (dd, J=7.6, 4.9 Hz, 1H); ¹³C NMR (126 MHz, CDCl₃, δ): 163.5, 152.8, 147.2, 130.1, 128.9, 128.5, 123.8, 121.1, 119.4, 116.1, 113.3; HRMS (ESI+): calculated for C₁₁H₇ClNO [M+H⁺], 204.0211; m/z found, 204.0212.

### 2-(3-Chloro-quinoxalin-2-yl)-4-fluoro-phenol

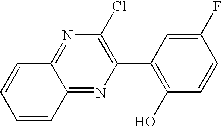

The title compound was prepared using methods analogous to those in Representative Procedure A. ¹H NMR (500 MHz, CDCl₃, δ) 11.1 (s, 1H), 8.30 (dd, J=8.9, 6.5 Hz, 1H), 8.09 (dd, J=7.6, 2.3 Hz, 1H), 8.05 (dd, J=7.2, 1.8 Hz, 1H), 7.85 (qd, J=7.0, 1.7 Hz, 1H), 7.83 (qd, J=7.5, 2.1 Hz, 1H), 6.85 (dd, J=10.2, 2.6 Hz, 1H), 6.77 (dd, J=8.0. 2.6 Hz, 1H); ¹³C NMR (126 MHz, CDCl₃, δ): 165.0 (d, J=252.2 Hz), 160.0 (d, J=13.1 Hz), 151.0, 141.1, 137.8, 137.1, 132.5 (d, J=10.6 Hz), 131.4, 131.2, 128.4, 127.6, 115.7 (d, J=3.1 Hz), 106.7 (d, J=22.2 Hz), 105.2 (d, J=24.2 Hz).

### 3-Fluoro-11-oxa-5,10-diaza-benzo[b]fluorine

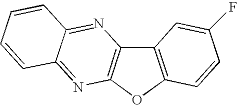

The title compound was prepared using methods analogous to those in Representative Procedure B. ¹H NMR (500 MHz, CDCl₃, δ) 8.30 (dd, J=8.6, 5.5 Hz, 1H), 8.28 (dd, J=6.0, 2.2 Hz, 1H), 8.13 (m, 1H), 7.81 (qd, J=8.9, 2.0 Hz, 1H), 7.81 (t, J=6.8 Hz, 1H), 7.40 (dd, J=8.5, 2.2 Hz, 1H), 7.25 (td, J=8.9, 2.2 Hz, 1H); ¹³C NMR (126 MHz, CDCl₃, δ): 165.2 (d, J=260.4 Hz), 159.2 (d, J 13.8 Hz), 156.0, 141.3, 139.7, 139.3, 129.7, 129.0, 128.6, 128.4, 124.1 (d, J=10.6 Hz), 117.6 (d, J=2.4 Hz), 112.8 (d, J=23.9 Hz), 101.0 (d, J=27.2 Hz); MS (EI+): calculated for C₁₄H₇FN₂O, 238.0; m/z found, 238.0 [M^{·+}].

### 2,4,8-Trichloro-benzo[4,5]furo[3,2-d]pyrimidine

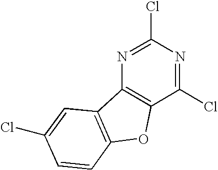

The title compound was prepared using methods analogous to those in Representative Procedure B. ¹H NMR (500 MHz, CDCl₃, δ): 8.22 (d, J=2.1 Hz, 1H), 7.75 (dd, J=8.9, 2.1 Hz, 1H), 7.69 (d, J=9.0 Hz, 1H); MS (Cl): calculated for C₁₀H₃Cl₃N₂O, 271.9; m/z found, 272.0 [M^{·+}].

### 4-Chloro-2-(2,4,6-trichloro-pyrimidin-5-yl)-phenol

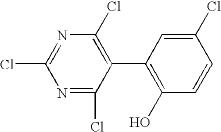

The title compound may be prepared using methods analogous to those described for Representative Examples D and E.

### 2,4,6-Trichloro-benzo[4,5]furo[2,3-d]pyrimidine

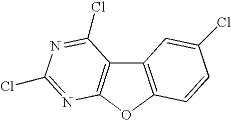

The title compound may be prepared using methods analogous to those described for Representative Example B.

While the invention has been illustrated by reference to examples, it is understood that the invention is intended not to be limited to the foregoing detailed description.

### REFERENCES

(1) Oliveira, A. M. A. G.; Manuela, M.; Raposo, M.; Oliveira-Campos, A. M. F.; Machado, A. E. H.; Puapairoj, P.; Pedro, M.; Nascimento, M. S. J.; Portela, C.; Afonso, C.; Pinto, M. Eur. J. Med. Chem. 2006, 41, 367-372.
(2) Voigt, B.; Meijer, L.; Lozach, O.; Schaechtele, C.; Totzke, F.; Hilgeroth, A. Bioorg. Med. Chem. Lett. 2005, 15, 823-825.
(3) Hurley, L. H.; Mahadevan, D.; Han, H.; Bearss, D. J.; Vankayalapati, H.; Bashyam, S.; Munoz, R. M.; Warner, S. L.; Della, C. K.; Von H., Daniel D.; Grand, C. L. PCT Int. Appl. (2005), WO 2005037825.
(4) Ishida, A.; Inage, M.; Akatsuka, H.; Inamasu, M.; mitsui, T. Jpn. Kokai Tokkyo Koho, JP 06220059, 1994.
(5) Harris, N.; Higgs, C.; Wren, S.; Dyke, H. J.; Price, S.; Cramp, S. PCT Int. Appl. (2006), WO 2006050965.
(6) Abramovitch, R. A.; Inbasekaran, M. N.; Kato, S.; Radzikowska, T. A.; Tomasik, P. J. Org. Chem. 1983, 48, 690-695.
(7) Yue, W. S.; Li, J. J. Org. Lett. 2002, 13, 2201-2203.
(8) Kawaguchi, K.; Nakano, K.; Nozaki, K. J. Org. Chem. 2007, 72, 5119-5128.
(9) (a) Martinez, A.; Fernandez, M.; Estevez, J. C.; Estevez, R. J.; Castedo, L. Tetrahedron 2005, 61, 1353-1362; (b) Litinas, K. E.; Nicolaides, D. N. J. Chem. Soc., Perkin Trans. 1 1985, 3, 429-435; (c) Brown, P. J. N.; Stephens, R.; Tatlow, J. C. Tetrahedron 1967, 23, 4041-4045.
(10) Parmintier, M.; Gros, P.; Fort, Y. Tetrahedron 2005, 61, 3261-3269.
(11) Coates, W. J.; McKillop, A. J. Org. Chem. 1990, 55, 5418-5420.
(12) (a) Zhang, S.; Zhang, D.; Liebeskind, L. S. J. Org. Chem. 1997, 62, 2312-2313; (b) Allred, G. D.; Liebeskind, L. S. J. Am. Chem. Soc. 1996, 118, 2748-2749; (c) Innitzer, A. Synlett 2005, 15, 2405-2406 and references cited therein.
(13) For isolated literature examples of halogen-selective Suzuki coupling between 1,2-dihalobenzenes and α-hydroxyphenylboronic acid derivatives, see: (a) Sinclair, D. J.; Sherburn, M. S. J. Org. Chem. 2005, 70, 3730-3733; (b) Buchwald, S. L.; Huang, X.; Zim, D. PCT Int. Appl. Publ. WO 2004/052939 A2; (c) Buchwald, S. L.; Old, D. W.; Wolfe, J. P.; Palucki, M.; Kamikawa, K. U.S. Pat. No. 6,307,087 B1.
(14) α-Hydroxyphenylboronic acids with other phenol protecting groups such as MOM can also be used if the functional groups are not stable under these demethylation conditions.
(15) Halogen-selective Suzuki couplings between 1,2-dihalopyrazines and α-hydroxyphenylboronic acids have been reported: Mao, L.; Sakurai, H.; Hirao, T. Synthesis 2004, 15, 2535-2539.
(16) For certain substrates, the reaction rate and yield can be enhanced by using more active phosphine ligands such as ^{t}Bu₃P. However, this can lead to lower regioselectivity or chemoselectivity since coupling at the undesired halogenated position was often observed.
(17) For a review on Ullmann coupling, see: Hassan, J.; Sevignon, M.; Gozzi, C.; Schulz, E.; Lemaire, M. Chem. Rev. 2002, 102, 1359-1469.

What is claimed is:

1. A process for preparing a compound of Formula (I) or salts thereof: 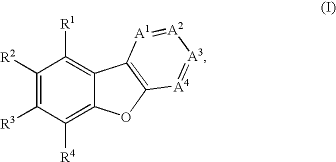
wherein
R¹, R², R³, and R⁴ are each independently H, fluoro, chloro, bromo, C₁₋₄alkyl, C₁₋₄alkoxy, —CF₃, —OCF₃, —CN, —NO₂, —S(O)C₁₋₄alkyl, —SO₂C₁₋₄alkyl, —CHO, —C(O)C₁₋₄alkyl, —CO₂C₁₋₄alkyl, —CO₂H, —C(O)NR^{a}R^{b}, or —NR^{a}R^{b};
where R^{a} and R^{b} are each independently C₁₋₄alkyl;
A¹, A², A³, and A⁴ are each independently CR^{c} or N;
where at least two of A¹⁻⁴ are CR^{c}; and
each R^{c} is independently H, fluoro, chloro, bromo, C₁₋₄alkyl, C₁₋₄alkoxy, —CF₃, —OCF₃, —CN, —NO₂, —S(O)C₁₋₄alkyl, —SO₂C₁₋₄alkyl, —CHO, —C(O)C₁₋₄alkyl, —CO₂C₁₋₄alkyl, —CO₂H, —C(O)NR^{d}R^{e}, or —NR^{d}R^{e};
where R^{d} and R^{e} are each independently C₁₋₄alkyl;
or two adjacent R^{c} groups taken together with the carbon members to which they are attached form a fused benzo ring; comprising
intramolecularly cyclizing a compound of formula (II): 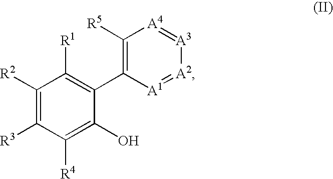
wherein R⁵ is chloro or bromo;
in the presence of a copper(I) salt, in a polar, aprotic organic solvent.

2. The process according to claim 1, wherein the cyclization is promoted by at least one molar equivalent of a copper(I) salt.

3. The process according to claim 2, wherein the copper(I) salt is copper(I) thiophene-2-carboxylate, CuCl, CuBr, CuOAc, or a mixture thereof.

4. The process according to claim 2, wherein the copper(I) salt is copper(I) thiophene-2-carboxylate.

5. The process according to claim 1, wherein the cyclization is performed in N,N-dimethylacetamide or N-methylpyrrolidone.

6. The process according to claim 1, wherein each of A¹⁻⁴ is CR^{c} and said cyclizing is performed at a temperature from about 80° C. to about 140° C.

7. The process according to claim 1, wherein at least one of A¹⁻⁴ is N and said cyclizing is performed at a temperature from about 40° C. and to 140° C.

8. The process according to claim 1, wherein R¹ is H; R² is H, chloro, or fluoro; R³ is H or fluoro; R⁴ is H; and each R^{c} is independently H, chloro, CF₃, CO₂H, or NO₂.

9. The process according to claim 1, wherein the compound of Formula (I) is selected from the group consisting of:
dibenzofuran;
2-chloro-8-fluoro-dibenzofuran;
dibenzofuran-2-carboxylic acid;
2-chloro-7-nitro-dibenzofuran;
benzo[4,5]furo[2,3-b]pyridine;
benzo[4,5]furo[2,3-c]pyridine;
benzo[4,5]furo[3,2-c]pyridine;
8-chloro-3-trifluoromethyl-benzo[4,5]furo[3,2-b]pyridine;
6-chloro-benzo[4,5]furo[2,3-b]pyridine;
3-fluoro-11-oxa-5,10-diaza-benzo[b]fluorine;
2,4,8-trichloro-benzo[4,5]furo[3,2-d]pyrimidine; and
2,4,6-trichloro-benzo[4,5]furo[2,3-d]pyrimidine.

10. The process according to claim 1, further comprising reacting a compound of formula (III): 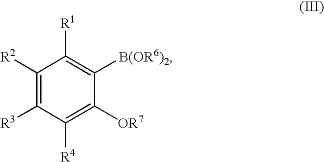
with a compound of formula (IV): 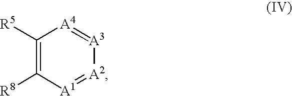
in the presence of a palladium(II) or palladium(0) catalyst and a ligand, and in the presence of a base, in a polar organic solvent;
to form a compound of formula (II);
wherein
R⁶ is H or C₁₋₄alkyl; or two R⁶ groups taken together form —C(CH₃)₂—C(CH₃)₂—;
R⁷ is H, C₁₋₄alkyl, methoxymethyl, (2-methoxyethoxy)methyl, benzyl, benzyloxymethyl, p-methoxybenzyl, trimethylsilyl, triethylsilyl, tert-butyldimethylsilyl, tert-butyldiphenylsilyl, or triisopropylsilyl; and
R⁸ is chloro, bromo, or iodo, when R⁵ is chloro, and R⁸ is bromo or iodo when R⁵ is bromo.

11. The process according to claim 10, wherein each R⁶ is H or methyl, or two R⁶ groups taken together form —C(CH₃)₂—C(CH₃)₂—.

12. The process according to claim 10, wherein R⁷ is H or methyl.

13. The process according to claim 10, wherein the palladium(II) catalyst is Pd(OAc)₂, PdCl₂, or a mixture thereof.

14. The process according to claim 10, wherein the palladium(0) catalyst is Pd(PPh₃)₄, Pd₂(dba)₃, or a mixture thereof.

15. The process according to claim 14, wherein the ligand is dppf, PPh₃, (tBu)₃P, (Chx)₃P, or a mixture thereof.

16. The process according to claim 10, wherein the base is K₃PO₄, KOH, K₂CO₃, Cs₂CO₃, Et₃N, NaOH, Na₃PO₄, Na₂CO₃, or a mixture thereof.

17. The process according to claim 10, wherein the polar organic solvent is acetonitrile, toluene, DMF, DME, THF, MeOH, EtOH, water, or a mixture thereof.

18. The process according to claim 10, wherein the palladium(II) catalyst is Pd(OAc)₂, the base is K₃PO₄, and the polar organic solvent is acetonitrile.

19. The process according to claim 10, further comprising deprotecting a compound of formula (IIa): 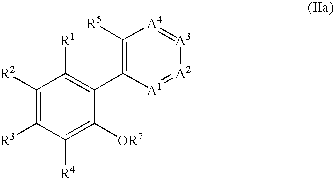
to form a compound of formula (II).

20. The process according to claim 19, wherein R⁷ is methyl and said deprotecting occurs in the presence of BBr₃.
